(19) United States
(12) Patent Application Publication
(10) Pub. No.: US 2005/0112760 A1
(43) Pub. Date: May 26, 2005

(54) In vitro culture of tissue structures

(75) Inventors: Syed H. Kamil, Shrewsbury, MA (US); Roland D. Eavey, Brookline, MA (US); Martin P. Vacanti, Westborough, MA (US)

Correspondence Address: FISH & RICHARDSON PC, 225 FRANKLIN ST, BOSTON, MA, 02110, US

(73) Assignee: Massachusetts Eye & Ear Infirmary, Boston, MA (US)

(21) Appl. No.: 10/898,468

(22) Filed: Jul. 23, 2004

Related U.S. Application Data
(63) Continuation of application No. 10/675,511, filed on Sep. 30, 2003.
(60) Provisional application No. 60/414,674, filed on Sep. 30, 2002.

(51) Int. Cl. C12N 5/08

(52) U.S. Cl. 435/366

(57) ABSTRACT

A method for growing a tissue structure includes providing a scaffold having the shape of the tissue structure and seeding the scaffold with tissue precursor cells. The seeded scaffold is then grown in vitro under conditions and for an incubation time sufficient for the tissue precursor cells to produce tissue on the scaffold and to from a tissue structure that can be implanted into a patient without causing an immune response.

### CROSS-REFERENCE TO RELATED APPLICATIONS

This application is a continuation application of and claims priority to U.S. Application Ser. No. 10/675,511, filed on Sep. 30, 2003, which claims priority from U.S. Provisional Application Ser. No. 60/414,674, filed Sep. 30, 2002. The contents of the prior applications are incorporated by reference herein.

### FIELD OF INVENTION

The invention relates to reconstructive surgery, and in particular, to growth of tissue structures, such as cartilaginous structures, for use in reconstructive surgery.

### BACKGROUND

Reconstructive surgery of the ear either requires autologous rib cartilage grafts or prosthetic devices. Reconstructive nasal surgery involves septal correction, separation of alar and upper lateral cartilages from the septum, osteotomies and possibly autogenous cartilage grafts for reconstruction and reinforcement of the nasal skeleton. These conventional procedures are associated with limited tissue availability, dissimilarity of the donor tissue, donor site morbidity and potential problems of resorption and distortion of the graft itself.

Tissue engineering involves the regeneration of tissues such as bone and cartilage by seeding cells onto a customized biodegradable polymer scaffold to provide a three-dimensional environment that promotes matrix production. This scaffold anchors cells and permits nutrient and gas exchange with the surrounding medium. The scaffold enables formation of new tissue in the shape of the polymer material. See, e.g., Vacanti et al., 1994, Transplant. Proc., 26:3309-3310; and Puelacher et al., 1994, Biomaterials, 15:774-778.

Using known tissue engineering techniques, cartilage can be generated successfully both in vitro and in vivo by using animal or human chondrocytes. Unfortunately, limitations still exist with current cartilage generation systems. For example, an ear supported with a scaffold of polyglycolic acid (hereafter referred to as “PGA”) and polylactic acid (hereafter referred to as “PLA”) can evoke an inflammatory response when implanted into an animal or human patient having a competent immune system.

Cartilage can be generated by seeding chondrocytes onto the three-dimensional scaffolds in vivo. However, after an initial success in a xenograft model, a significant challenge has been to generate cartilage by using an in vivo autologous model that maintains a refined shape. Known autologous models use a PGA/PLA scaffold, such as those described in U.S. Pat. No. 5,770,193, U.S. Pat. No. 5,736,372, U.S. Pat. No. 6,171,610, the contents of which are herein incorporated by reference. Such scaffolds, however, tend to evoke an inflammatory reaction in an immunocompetent animal model. A more robust cartilage can be provided by a gel skeleton; however, the resulting shape is suboptimal.

### SUMMARY OF THE INVENTION

The invention is based on the discovery that artificial tissue structures or constructs can be created using certain biodegradable and/or non-biodegradable materials as a scaffold, seeding the scaffold with tissue precursor cells, and then culturing the seeded scaffold in vitro under conditions and for a time sufficient to obtain a tissue structure that does not cause an immune response when implanted into a patient, e.g., a human or animal patient.

In one aspect, the invention includes a method for growing a tissue structure by providing a scaffold having the shape of the tissue structure. The scaffold, which includes a biodegradable and/or non-biodegradable material, is seeded with tissue precursor cells. The seeded scaffold is then grown in vitro under conditions and for an incubation time sufficient for the tissue-producing cells to produce tissue on the scaffold.

Practices of the invention include those in which the scaffold includes non-biodegradable acrylic, polyethylene, polypropylene, polystyrene, or purified silastic. Inert, non-immunogenic metals such as gold can also be used. The non-biodegradable materials must be immunologically inert. The biodegradable materials need not be completely immunologically inert, but must be able to degrade and dissolve within the culture time, e.g., within 5, 8, 10, or 12 weeks, or 4, 5, 6, 7, or 8 months in culture. Such materials include, e.g., polyglycolic acid.

Where a cartilaginous structure is to be grown, the tissue precursor cells include chondrocytes. In general, “tissue precursor cells” are cells that form the basis of new tissue. Tissue cells can be “organ cells,” which include hepatocytes, islet cells, cells of intestinal origin, muscle cells, heart cells, cartilage cells, bone cells, kidney cells, cells of hair follicles, cells from the vitreous humor in the eyes, cells from the brain, and other cells acting primarily to synthesize and secret, or to metabolize materials. In some embodiments, these cells can be fully mature and differentiated cells. In addition, tissue precursor cells can be so-called “stem” cells or “progenitor” cells that are partially differentiated or undifferentiated precursor cells that can form a number of different types of specific cells under different ambient conditions, and that multiply and/or differentiate to form a new tissue.

An “isolated” tissue precursor cell, such as an isolated chondrocyte, or an isolated chondrocyte stem or progenitor cell, or bone cell, or bone stem or progenitor cell, is a cell that has been removed from its natural environment in a tissue within an animal, and cultured in vitro, at least temporarily. The term covers single isolated cells, as well as cultures of isolated cells, e.g., cultures that have been significantly enriched for the cells, e.g., stem or progenitor cells, with few or no differentiated cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The ability to generate cartilage and other tissues having a complex structure, such as that of the external ear and nasal tip, is an important step in the generation of engineered tissue constructs. Such detailed constructs are of particular usefulness in reconstructive surgery, particularly where a patient's cartilaginous skeleton has been distorted or destroyed due to, for example, congenital deformity, post-operative complication, trauma, granulamotous disease, or malignancy.

Other features and advantages of the invention will be apparent from the following detailed description, and from the claims.

### BRIEF DESCRIPTION OF THE DRAWINGS

FIG. 1 is an anterior view of a nasal tip tissue construct.

FIGS. 2 and 3 are views of the nasal tip of FIG. 1 from above and below, respectively.

FIG. 4 is a view of an ear tissue construct grown in vitro.

FIG. 5 is a view of an ear tissue construct grown in vitro after having been implanted into a rat, and then reviewed.

### DETAILED DESCRIPTION

The invention provides a method for growing a tissue structure in vitro by seeding a scaffold with tissue precursor cells, such as chondrocytes. To grow a body part (tissue construct), a scaffold having a desired shape, for example the shape of an external ear or a nasal tip cartilage (alar, septal and upper lateral), is created by placing a biodegradable and/or non-biodegradable material into a mold. For example, one can place a mixture of 100 micron-thick sheets of polyglycolic acid (PGA) fibers and poly-L-lactic acid (hereafter referred to as “PLLA”) into silicone molds having the desired shape. For auricular tissue constructs and other constructs that require some rigidity, a non-biodegradable material, such as acrylic sheets, can be embedded into the biodegradable material, e.g., PGA fibers. A suitable source of PGA fibers is Davis & Geck, of Danbury, Conn. Suitable acrylic sheets are available from Alcon Research, of Fort Worth, Tex.

The idea is to have any scaffold materials that are at all immunogenic to be totally biodegradable, and to use only non-immunogenic materials for any non-biodegradable portions of a scaffold. The combination provides a scaffold in which the biodegradable (potentially immunogenic) portions are dissolved and replaced over time with cartilage or other tissue (such as bone or fibrous tissue), and in which the non-biodegradable portions need not be replaced (since they are non-immunogenic), but remain to provide long-term support of the desired rigidity in the final tissue construct.

PGA is a highly porous and biodegradable polymer with interfiber spaces of 75 to 100 microns. As such, it forms a mesh that is unable to retain a specific shape on its own. PLLA dissolved in methylene chloride stiffens this mesh by providing cross-linkage of PGA fibers. A 1.5% solution of PLLA in methylene chloride achieves rigidity of the scaffold without compromising its porosity. The methylene chloride is allowed to evaporate.

Biodegradable scaffolding materials other than PGA fibers can also be used. Examples of such scaffolding materials include Pluronic F-127, calcium alginate, or collagen gel. Non-biodegradable scaffold materials include acrylic, gold, high-density polyethylene, and extrapurified Silastic. Others can be used as long as they are truly non-immunogenic after long-term (e.g., more than 1 month) implantation into an immunocompetent mammal. See, e.g., Arevalo-Silva et al., Arch. Otol. Head Neck Surg., 126:1448-1452 (2000).

Once dried, the scaffolds are sterilized by immersion in 70% ethanol for 20 minutes. The ethanol is then completely aspirated to again dry the scaffolds. This procedure is repeated to optimize sterilization. Finally, the scaffolds are immersed in Ham's F-12 culture medium for 30 minutes to eliminate any residual alcohol. The scaffolds are then air-dried completely.

Next, tissue precursor cells are seeded into or onto the completed scaffold. Chondrocytes, for example, can be derived from fresh cartilage, which can be obtained from newborn calf shoulders, preferably within six hours of slaughter. The cartilage is harvested under aseptic conditions from the shoulder joints; fragmented into small pieces; and washed in a phosphate-buffered saline (PBS) solution containing antibiotics for 8-12 hours. A suitable mixture of antibiotics includes 100 ug/L of penicillin, 100-mg/L of streptomycin and 0.25-mg/L of amphoterecin B obtained from Gibco, Grand Island, N.Y. The mixture is then digested with 0.3% collagenase II, which can be obtained from Worthington Biochemical Corporation of Freehold, N.J.

The resulting cell suspension is passed through a sterile 180 micron filter, such as the Spectra/Mesh 146-426 obtainable from Spectrum Medical Industries of Laguna Hills, Calif. The filtrate is then centrifuged and the resulting cell pellet is washed twice with copious amounts of Dulbecco's phosphate-buffered saline. Cell number and viability are then determined by cell count using a hemocytometer and tryphan blue dye.

Chondrocyte suspensions are created by mixing the cells with Ham's F12 culture medium to a cellular density of 50 million cells/ml. Chondrocytes are seeded onto the scaffolds, both by coating the outside of the scaffolds with chondrocytes and by injecting chondrocytes into the scaffolds using a 16-gauge needle.

Once seeded, the scaffolds must be cultured under conditions and for a time sufficient for the scaffold materials that have any immunogenic components to be degraded and removed from the tissue construct. The non-immunogenic scaffold materials, such as acrylic sheets, fibers, or rods, can remain in the tissue construct. It is important that the culture conditions permit the tissue precursor cells to survive and thrive for many weeks or months, and to avoid long-term culture problems, such as mold, bacterial, or viral infection.

For example, scaffolds seeded with chondrocytes are placed in a Petri dish for three days in Ham's F-12 culture medium, which can be obtained from Life Technologies of Baltimore, Md. The medium also contains L-glutamine, 50 mg/L L-ascorbic acid, 100 u/L of penicillin, 100-mg/L of streptomycin, 0.25-mg/L of amphoterecin B, supplemented with 10% fetal bovine serum, such as that obtained from Sigma-Aldrich of St. Louis, Mo. The cell cultures are maintained at 37 degrees C. and 5% CO₂. The culture medium is changed twice daily for the first three days. The frequent changing of the culture medium promotes an aerobic environment that enhances formation of larger cartilaginous structures.

An alternative practice of the invention replaces the bovine serum with human serum or with growth factors. This avoids contact with bovine serum. The structures grown in this way are thus more suitable for implantation in humans. Alternatively, the chondrocytes are human chondrocytes that can be collected from a compatible donor or from the patient.

Once the tissue precursor cells, e.g., chondrocytes, have attached to the scaffold, e.g., PGA fibers, the scaffolds are put into long-term culture vessels under long-term culture conditions. For example, cultured chondrocyte-seeded scaffolds can be placed in small bottles on a continuous gentle shaker inside an incubator. The chondrocytes incubate for several weeks, during which time the culture medium is changed twice a week. Strict aseptic conditions are maintained while handling these bottles.

After several weeks of incubation, the scaffolds are examined for shape, structural details, and firmness by gentle palpation. Inspections can be carried out after eight, ten, and twelve weeks of growth. Typically, the scaffolds are ready to be removed from the in vitro culture after about twelve weeks. The main point is that any scaffold materials that may be or include any immunogenic components be totally dissolved and removed from the tissue construct while maintaining tissue cells alive in the tissue construct.

After twelve weeks of in vitro growth, cartilage progressively replaces the polymer fibers. As this occurs, the construct becomes progressively more rigid. A cartilaginous skeleton having alar, septal, and upper lateral cartilages replaces the nasal tip scaffolds. Similarly, a cartilaginous skeleton in the shape of a human ear replaces the auricular scaffolds.

For auricular constructs, a more natural external shape, better structural details, and a proper firmness of the tissue construct are obtained by providing internal support with a permanent, non-biodegradable scaffold material, such as with soft acrylic sheets, rods, or fibers. These constructs maintain their exact contours after subcutaneous implantation for an additional six weeks.

Acrylic is an advantageous material because it is already approved for intra-ocular implants. However, other non-degrading materials can be used in place of acrylic for providing internal support, either in an auricular construct or any other type of construct. Examples of such materials include polyethylene, polypropylene, and polystyrene. Even gold rods, sheets, or fibers can be used. The non-degrading material can be made to retain its shape by crumpling it or by stamping it in a mold having the desired shape.

Culture conditions and duration of in vitro cultivation can modulate the mechanical properties of tissue-engineered cartilage. During prolonged in vitro culture, the weight fraction of glycosaminoglycans in tissue-engineered constructs approaches that of fresh cartilage. The firmness of the constructs, as judged by gentle palpation, increases with the duration of in vitro culture. Gentle rotation of the culture bottles appears to enhance the secretion of an extracellular matrix.

Samples can then be obtained for histological analysis. Once fixed for at least 24 hours, the samples are embedded in paraffin and sectioned using standard histochemical techniques. Slide sections are stained with hematoxylin and eosin, and Safranin-O stains. Biochemical analysis can then be performed to confirm the formation of hydroxyproline and glycosaminoglycans. Auricular constructs with internal support are then implanted into a live animal for six weeks to determine continued survival in vivo. The constructs are removed from the animal aseptically and frozen at −86 degrees centigrade.

A histological examination of a typical construct made according to the process described herein, when stained with hematoxylin and eosin, reveals areas of cartilage mixed with fibrous tissue. The matrix is heterochromatic with both light basophilia and eosinophlia interspersed with round to oval lacunae containing mainly chondrocytes. A positive Safranin-O stain demonstrates proteoglycans content.

The method of the invention thus consistently generates the delicate cartilaginous skeletons of the external ear and nasal tip structures, or of any desired shape. Full sized human auricular and nasal tip cartilaginous skeleton (alar, septal and upper lateral) can be generated using only in vitro tissue engineering techniques.

Cartilage is a specialized type of dense connective tissue consisting of cells embedded in a matrix. There are several kinds of cartilage. Hyaline cartilage is a bluish-white, glassy translucent cartilage having a homogeneous matrix containing collagenous fibers that is found in articular cartilage, in costal cartilages, in the septum of the nose, and in the larynx and trachea. Articular cartilage is hyaline cartilage covering the articular surfaces of bones. Costal cartilage connects the true ribs and the sternum. Fibrous cartilage contains collagen fibers. Yellow cartilage is a network of elastic fibers holding cartilage cells which is found primarily in the epiglottis, the external ear, and the auditory tube. By harvesting the appropriate chondrocyte precursor cells, any of these types of cartilage tissue can be grown using the methods of the invention.

Cells other than chondrocytes can be grown using methods similar to those described herein. Examples of such cells include other cartilage producing cells, macrophages, adipocytes, dermal cells, muscle cells, hair follicles, fibroblasts, organ cells, osteoblasts, osteocytes, and other cells that form bone, endothelial cells, mucosal cells, pleural cells, ear canal cells, tympanic membrane cells, peritoneal cells, Schwann cells, corneal epithelial cells, gingival cells, central nervous system neural stem cells, or tracheal epithelial cells.

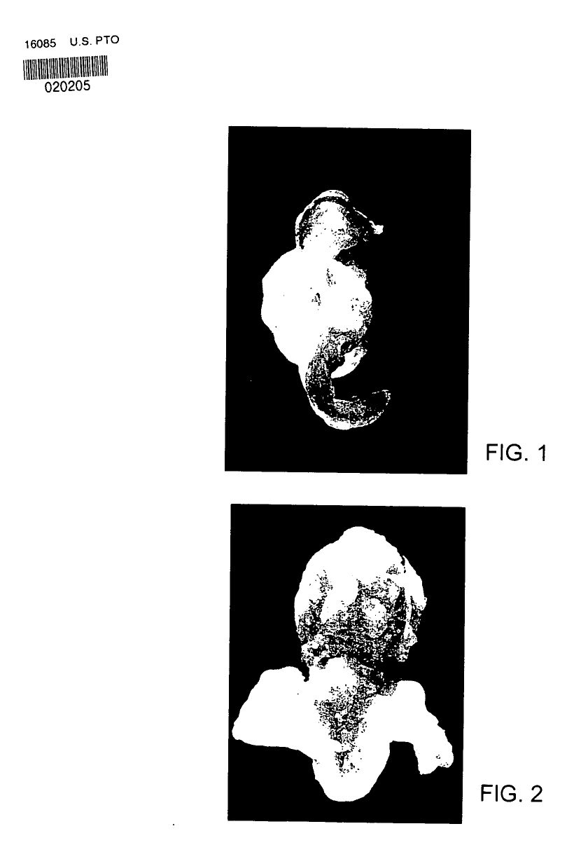

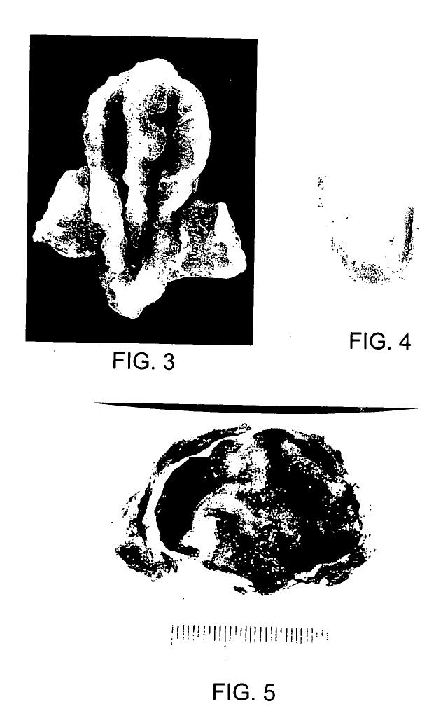

The figures show cartilaginous structures made using the method of the invention. In particular, FIG. 1 shows an anterior view a nasal tip. FIGS. 2 and 3 show the nasal tip of FIG. 1 from above and below respectively. FIG. 4 shows an ear in vitro. FIG. 5 shows an in vitro ear grown in a rat.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for growing a tissue structure, the method comprising:
providing a scaffold having the shape of the tissue structure, the scaffold including a biodegradable and/or non-biodegradable material;
seeding the scaffold with tissue precursor cells; and
growing the seeded scaffold in vitro under conditions and for an incubation time sufficient for the tissue precursor cells to produce tissue on the scaffold and form a tissue structure that can be implanted into a patient without causing an immune response.

2. The method of claim 1, further comprising selecting the scaffold to include one or more materials selected from the group consisting of acrylic, polyethylene, polypropylene, and polystyrene.

3. The method of claim 1, further comprising selecting the scaffold to include a plastic.

4. The method of claim 1, further comprising selecting the tissue precursor cells to include chondrocytes.

5. The method of claim 1, further comprising selecting the incubation time to be at least ten weeks.

6. A tissue structure grown by the method of claim 1.
